(19) United States
(12) Patent Application Publication
(10) Pub. No.: US 2020/0222325 A1
(43) Pub. Date: Jul. 16, 2020

(54) METHOD FOR PREPARING AT LEAST PARTIALLY DRIED GRANULES

(71) Applicant: NOVARTIS AG, Basel (CH)

(72) Inventors: Markus KRUMME, Allschwil (CH); Hans DE WAARD, Basel (CH); Klaus-Peter MOLL, Basel (CH); Adrian SCHMIDT, Weil am Rhein (DE); Frantz ELBAZ, Rixheim (FR); Peter KLEINEBUDDE, Düsseldorf (DE)

(21) Appl. No.: 16/640,756

(22) PCT Filed: Aug. 30, 2018
(86) PCT No.: PCT/IB2018/056631

§ 371 Date: Feb. 21, 2020

(30) Foreign Application Priority Data
Aug. 31, 2017 (EP) 17188804.3

(51) Int. Cl. A61K 9/16; A61K 45/06; A61K 9/20; A61K 9/48

(52) U.S. Cl. CPC A61K 9/1694; A61K 9/4833; A61K 9/2095; A61K 45/06

(57) ABSTRACT

In a method for preparing at least partially dried granules, an API, a base powder, and a granulation liquid are fed to a granulator. The API, the base powder and the granulation liquid are mixed within the granulator to produce an API/base powder/granulation liquid mixture. This liquid mixture within the granulator is heated to a heating temperature (T_{H}) that exceeds an evaporation temperature (T_{E}) of the granulation liquid contained in an API/base powder/granulation liquid mixture to allow at least a part of the granulation liquid contained in the API/base powder/granulation liquid mixture to evaporate in order to obtain at least partially dried granules containing at least the API and the base powder. The at least partially dried granules are discharged from the granulator.

The invention relates to a method for preparing at least partially dried granules. Further, the invention relates to at least partially dried granules prepared by a method of this kind. Finally, the invention relates to a method for manufacturing solid oral dosage forms containing dried granules of this kind.

Active pharmaceutical ingredients (API) intended for oral dosage typically undergo a number of synthesizing and washing steps in a liquid and finally are dried, for example by evaporation separation, in order to isolate the pure API from the auxiliary liquid phase used during the crystallization and washing steps. Thereafter, the primary API powder particles are further processed in order to form granules, i.e. multi-particulate entities suitable either for direct oral dosage or for further processing in order to prepare oral dosage forms such as tablets or capsules. Wet granulation of the dried pure API is a widely applied process step to enlarge particle sizes, to transform the properties of the API to enhance flow, compressibility, dissolution and hence biological exposure of the formulated compound and/or to dilute the concentration of the pure API to enable a volumetric dosing of the API during later processing steps.

For example, the dried pure API may be mixed with a binder and/or a granulation liquid such as water or an aqueous binder solution and thereafter again dried. In particular high-shear mixers with subsequent fluid bed drying or fluid bed dryers with the granulation liquid being sprayed into a fluidized powder bed may be used for producing dry granules that consist of the primary API particles bound together by a binding agent. These dry granules may already be suitable for oral dosage or can be further processed into a desired solid oral dosage form. Alternatively, the API and suitable excipients may be fed as dry solid particles to a screw granulator. A granulation liquid may be added separately into the granulator and after extensive mixing and due to applied shear, wet granules are obtained. These wet granules are dried and thereafter again may already be suitable for oral dosage or can be further processed into a desired solid oral dosage form. In any case, following the granulation step, a separate drying step, which is performed in a drying equipment, is required in order to remove excess liquid from the granules prepared during the granulation step.

The invention is directed at the object of providing a method and a granulator which allow an efficient preparation of at least partially dried granules. Further, the invention is directed at the object of providing a method for manufacturing solid oral dosage forms containing at least partially dried granules of this kind.

This object is addressed by a method for preparing at least partially dried granules as defined in claim 1, a granulator for preparing at least partially dried granules as defined in claim 11 and a method for manufacturing solid oral dosage forms as defined in claim 15.

In a method for preparing at least partially dried granules, an API, a base powder and a granulation liquid are fed to a granulator. The API may be any API that is suitable for being processed so as to form granules and may contain at least one of ibuprofen, lumenfantrine, and LDK378. Basically, the base powder may be an excipient and may contain a binder. The addition of a binder allows the formation of granules, i.e. powder agglomerates from primary API particles. The base powder may consist exclusively of a single binder or a binder mixture. It is, however, also conceivable that the base powder contains further additives for tailoring the properties of the API/base powder mixture and/or the resulting granules as desired. In particular, the base powder may contain at least one of (spray dried) lactose, cellulose, in particular microcrystalline cellulose, sodium carboxymethyl cellulose, glyceryl behenate, magnesium stearate, sodium stearyl fumarate, polyvinylpyrrolidone, sodium pyrophosphate, and calcium phosphate. The base powder may, however, also contain or consist of solid API powder particles. In particular, the base powder may contain powder particles of the same API that is fed to the granulator anyway. As a result, more or less pure API granules may be obtained.

The API, the base powder and the granulation liquid are mixed within the granulator in order to produce an API/base powder/granulation liquid mixture. The granulation liquid may be any liquid that is suitable for producing an API/base powder/granulation liquid mixture that can be further processed so as to form granules. For example, water or an organic solvent, in particular ethanol, methanol, isopropanol and ethylacetate can be used as the granulation liquid.

The API/base powder/granulation liquid mixture is heated within the granulator to a heating temperature that exceeds an evaporation temperature of the granulation liquid contained in the API/base powder/granulation liquid mixture so as to allow at least a part of the granulation liquid contained in the API/base powder/granulation liquid mixture to evaporate in order to obtain at least partially dried granules containing at least the API and the base powder. Finally, the at least partially dried granules are discharged from the granulator.

In other words, in the method for preparing at least partially dried granules, drying of the API/base powder/granulation liquid mixture takes place already in the granulator. Preferably, the at least partially dried granules resulting from this in situ drying process contain not more than 10% residual moisture. Typical sizes of the at least partially dried granules vary from 200 to 1000 μm. Further, the dried granules provide for the desired biological exposure of the API, i.e. the desired dissolution of the API in the body of a patient. In dependence on the type of API and the desired route of administration, the at least partially dried granules obtained by the method described herein may be intended for direct oral dosage or may be intended for further processing into a desired administration form.

In the method for prepared at least partially dried granules, a separate drying step following the granulation process, which typically is performed in a separate drying equipment, can be dispensed with or at least significantly shortened. Consequently, the efficiency of the granulation and drying process is significantly increased.

The API/base powder/granulation liquid mixture may be heated to a heating temperature that is between 1.5 to 2.5 times the evaporation temperature of the granulation liquid. This ensures that the major part of the granulation liquid, i.e. the liquid envelope surrounding the solid powder particles in the API/base powder/granulation liquid mixture, is removed so as to obtain granules that can be further processed without time-consuming further drying.

Preferably, the API/base powder/granulation liquid mixture is heated to the heating temperature for a heating time of <15 seconds, preferably <10 seconds, in particular <6 seconds. The heating time should be kept as short as possible in order to avoid a deterioration of the API. Thus, in particular the combination of a high heating temperature with a short heating time enables the desired evaporation and hence removal of the granulation liquid, while the powdery API particles remain cold and hence are not affected by the heating process.

Preferably, the API/base powder/granulation liquid mixture, prior to being heated to the heating temperature, is preheated to a preheating temperature that is lower than the heating temperature. Preferably, the preheating temperature is between 0.2 and 1.0 times the evaporation temperature of the granulation liquid contained in the API/base powder/granulation liquid mixture. A preheating of the API/base powder/granulation liquid mixture avoids a sudden evaporation of the granulation liquid and hence supports the continuity and uniformity of the granulation and drying process.

In particular, the API/base powder/granulation liquid mixture may be preheated to the preheating temperature for a preheating time of <15 seconds, preferably <10 seconds, in particular <6 seconds. Preferably, the preheating time is longer than the heating time in order to allow a smooth heating process to take place. In a preferred embodiment of the method, during preheating of the API/base powder/granulation liquid mixture, granulation is performed, whereas drying is still not targeted.

Basically, granulation liquid that evaporates from the API/base powder/granulation liquid mixture may be discharged from the granulator via a discharge opening that is provided for discharging the at least partially dried granules from the granulator. In a preferred embodiment of the method for preparing at least partially dried granules, at least a part of the granulation liquid that evaporates from the API/base powder/granulation liquid mixture, however, is discharged from the granulator via an evaporation opening that is formed separate from a discharge opening for discharging the at least partially dried granules from the granulator and separate from (a) feeding port(s) for feeding the API, the base powder and/or the granulation liquid to the granulator. In other words, the evaporating granulation liquid preferably is removed from the granulator via an evaporation opening that exclusively serves for this purpose. As a result, the evaporation process is not affected by either the supply of API, base powder and/or granulation liquid to the granulator or the discharge of at least partially dried granules from the granulator. If need be, for example in case the API/base powder/granulation liquid mixture contains a high volume content of granulation liquid or in case the evaporation process should be accelerated, a plurality of exclusive evaporation openings may be provided in the granulator and used for discharging the evaporating granulation liquid from the granulator.

The API/base powder/granulation liquid mixture may be processed prior to being heated, in particular for a processing time of 0 to 10 seconds. The processing time may be determined and adjusted in dependence on the amount of solid particles contained in the API/base powder/granulation liquid mixture. For example, the processing time may be reduced or even eliminated with a decreasing solid particle content in the API/base powder/granulation liquid mixture. An API/base powder/granulation liquid mixture with a low solid particle content and hence a high granulation liquid content needs less processing, i.e. mixing, but more time for evaporating and hence removing the granulation liquid from the API/base powder/granulation liquid mixture.

In a preferred embodiment of the method for preparing at least partially dried granules, the base powder is provided to the granulator in the form of substantially dry solid powder particles. Further, also the API may be fed to the granulator in the form of substantially dry solid powder particles. In the context of this application, the expression “substantially dry solid powder particles” should designate solid particles in powder form which to not contain more than 10% residual moisture.

It is, however, also conceivable that the API is fed to the granulator in the form of a slurry containing solid API powder particles dispersed in a liquid. In dependence on the type of the API and the properties of the API powder such as, for example, particle size, particle size distribution, surface properties, wetting properties, etc., a concentration of the API within the suspension may be between 10 to 70 weight %. For example, when using ibuprofen as the API, the concentration of the API within the suspension may be between 29 to 53 weight %, when using lumefantrine as the API, the concentration of the API within the suspension may be between 25 to 35 weight %, and when using Opadry yellow as a model API, the concentration of the model API within the suspension may be between 10 to 20 weight %. In any case, the flow properties of the suspension should be adjusted by appropriately selecting the API load in the suspension and the type of liquid used for preparing the slurry in such a manner that the slurry can be reliably fed to the granulator in a desired volumetric amount and with a desired feed rate. Within the granulator, the slurry may be mixed with the base powder.

By feeding the API to the granulator in the form of a slurry, fine gravimetric dosing of dry API powder can be dispensed with. Instead, the API can be reliably fed to the granulator in a desired amount by simply adjusting the API content, i.e. the API concentration within the slurry and the feed rate of the slurry to the granulator. Hence, time-saving and cost efficient continuous volumetric dosing of the API powder is made possible. In addition, mixing the API with the base powder with the

API particles being dispersed in a liquid, may reduce stress and shear applied to the API particles during mixing and hence may be beneficial for the quality of the API.

In the method for preparing at least partially dried granules, the liquid contained in the API loaded slurry may be the only granulation liquid that is used in the process. It is, however, also conceivable to add additional granulation liquid to the granulator if needed. The additional granulation liquid may be the same liquid as the liquid used for preparing the API loaded slurry and, for example, may contained water and/or an organic solvent, in particular ethanol. The liquid/solid ratio within the slurry/base powder mixture mainly depends on the API concentration and the targeted drug load in the final composition and may be between 0.4 to 2.0. For example, when using ibuprofen as the API, the liquid/solid ratio within the slurry/base powder mixture may be between 0.5 to 1.4, when using lumefantrine as the API, the liquid/solid ratio within the slurry/base powder mixture may approximately 0.6, and when using Opadry yellow as a model API, the liquid/solid ratio within the slurry/base powder mixture may be between 0.2 and 0.4. Basically, the solid content within the slurry/base powder mixture prior to the evaporation of the granulation liquid should already be as high as possible in order to reduce the heating time during which the API is exhibited to an elevated temperature and in order to shorten the evaporation process. On the other hand, the liquid content within the API/base powder/granulation liquid mixture should be high enough to allow the formation of granules and proper mixing of the solid API particles with the base powder so as to ensure homogeneous distribution of the API in the resulting granules.

Basically, in case the API is fed to the granulator in the form of an API loaded slurry, the liquid contained in the slurry may be any desired liquid which is added to a previously dried API powder. Preferably, however, the liquid contained in the slurry contains a synthesizing liquid used for synthesizing the solid API powder particles in a preceding API synthesizing step or a washing liquid used for washing the solid API powder particles in a preceding washing step. In case a liquid used in a preceding API synthesizing step or a preceding washing step is “reused” in the granules preparation process, drying of the API prior to granulation processing can be dispensed with. In other words, a step of drying the API powder after synthesizing and/or washing which is energy and time intensive and hence unattractive from a cost perspective is no longer necessary. Further, also additional corrective processing steps such as milling or sieving that might be required after drying of pure API powders in order to maintain the desired particle properties and/or to obtain an API powder with good flowability which is suitable for further processing may be omitted. Finally, exposing the pure API to the thermal stress of drying can be avoided. For example, a wet filter cake obtained by filtering an API containing washing liquid after the washing step may be used as the slurry or as a basis for the slurry which may be diluted with further (washing) liquid if needed. The wet filter cake may, e.g., contain approximately 50% of solid API powder particles.

The slurry may contain only solid API particles and a liquid phase consisting of a synthesizing liquid used for synthesizing the solid API powder particles in a preceding API synthesizing step and/or a washing liquid used for washing the solid API powder particles in a preceding washing step. It is, however, also conceivable to add further liquid or solid components to the slurry for tailoring slurry properties such as, for example, dispersion of the API particles in the liquid phase, wetting of the API particles by the liquid phase, viscosity of the slurry, etc.. For example, polysorbate, sodium pyrophosphate or another surfactant may be added to the slurry as a wetting agent.

In a preferred embodiment of the method for preparing at least partially dried granules, at least one of the API and the base powder is fed to the granulator in the form of substantially dry solid powder particles upstream of the granulation liquid. As a result, the substantially dry solid powder particles can be preprocessed in the granulator before the granulation liquid is added, wherein a retention time of the powder particles within the granulator without the granulation liquid being added, can be adjusted in dependence on the characteristics of the powder particles. The preprocessing of the substantially dry solid powder particles may, for example, include pre-compressing of the powder particles prior to the addition of the granulation liquid. For adjusting the preprocessing time of the powder particles, the conveying speed of the powder particles through the granulator and a distance between a first feeding port for feeding the substantially dry solid powder particles to the granulator and a second feeding port for feeding the granulation liquid to the granulator can be selected as needed.

In case both the API and the base powder are fed to the granulator in the form of substantially dry solid powder particles, a mixture of the API and the base powder may be fed to the granulator via a common first feeding port that is arranged upstream of the second feeding port for feeding the granulation liquid to the granulator. Alternatively, it is conceivable to employ a granulator having a plurality of first feeding ports, for example two first feeding ports, so that the powdery API and the powdery base powder can be fed to the granulator at separate first feeding ports. In case, however, the API is fed to the granulator in the form of a slurry, the dispersed API particles, together with the liquid contained in the slurry and optionally also an additional granulation liquid may be fed to the granulator via the second feeding port. Alternatively, it is conceivable to employ a granulator having a plurality of second feeding ports, for example two second feeding ports, so that the API slurry and an additional granulation liquid can be fed to the granulator at separate second feeding ports. The second feeding port(s) may comprise a feeding nozzle which allows the supply of the slurry and/or the granulation liquid to the granulator at a desired feed rate.

The feed rate of the substantially dry solid powder particles to the granulator and the feed rate of the slurry and/or the granulation liquid to the granulator may vary in dependence on the design and in particular the dimensions of the granulator, the flow properties of the substantially dry solid powder particles, the API load, i.e. the liquid/solid ratio within the slurry, the rheology, in particular the viscosity of the slurry and/or the granulation liquid, the liquid/solid ratio within the API/base powder/granulation liquid mixture, the rheology, in particular the viscosity of the API/base powder/granulation liquid mixture, the wetting behavior of the substantially dry solid powder particles with the liquid contained in the API loaded slurry and/or the granulation liquid, i.e. the required amount of liquid to form granules with favourable properties, etc.. Typical feed rates of the substantially dry powder particles to a small scale granulator with a 11 mm diameter screw may, e.g., vary between 140 and 500 g/h, whereas typical slurry feed rates to the small scale granulator may vary between 200 and 1000 g/h. Typical feed rates to a medium scale granulator with a 16 mm diameter screw may be up to 10 times higher and typical feed rates to a large scale granulator with a 47 mm diameter screw may be up to 50 kg/h.

In a preferred embodiment of the method for preparing at least partially dried granules, the granulation liquid, either in the form of a pure granulation liquid or in the form of an API loaded slurry, is fed to the granulator by means of a metered pump. By using a metered pump, a reliable continuous volumetric dosing of the granulation liquid to the granulator is made possible.

The substantially dry solid powder particles may be fed to the granulator by means of a powder dosing device which is controlled in dependence on a weight change of a reserve of the powder particles contained in a reservoir of the powder dosing device. The granulation process may be interrupted for refilling the reservoir of the powder dosing device with the substantially dry solid powder. It is, however, also conceivable to simply interrupt the weight change control and to maintain the feed rate of powder to the granulator constant, i.e. to operate the powder dosing device “blind” during refilling the reservoir.

In a method for manufacturing solid oral dosage forms granules are prepared in accordance with an above described method. The obtained granules are compacted so as to form tablets. Alternatively, the obtained granules are filled into capsules.

Preferred embodiments of the invention now will be described in greater detail with reference to the appended schematic drawings and examples, wherein:

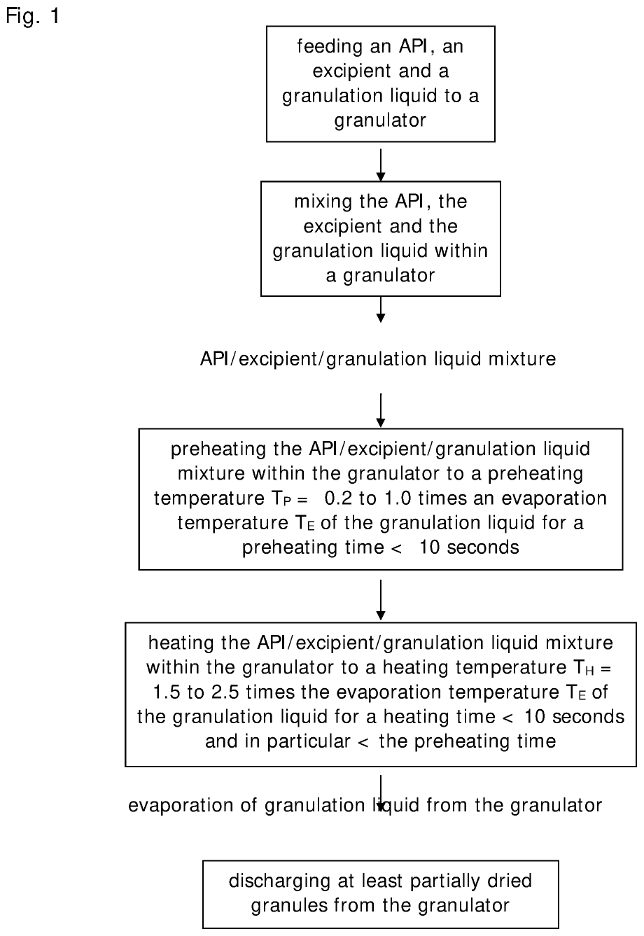

FIG. 1 shows a flow diagram depicting a method for preparing at least partially dried granules,

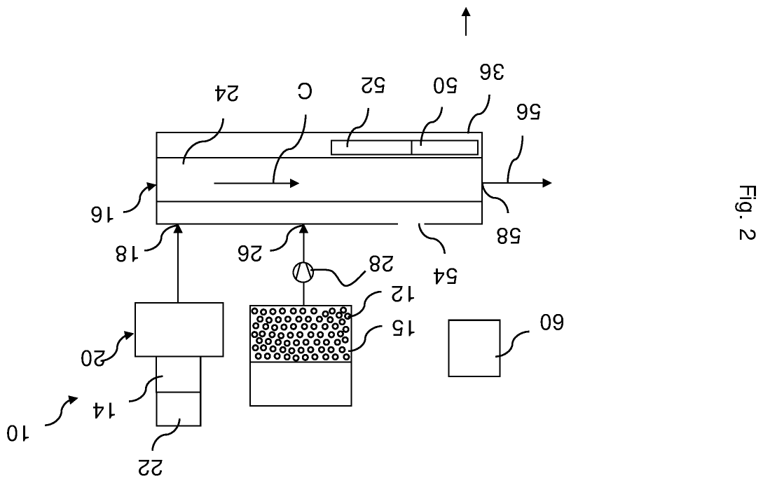

FIG. 2 shows a layout of a system for preparing granules in accordance with the method of FIG. 1, and

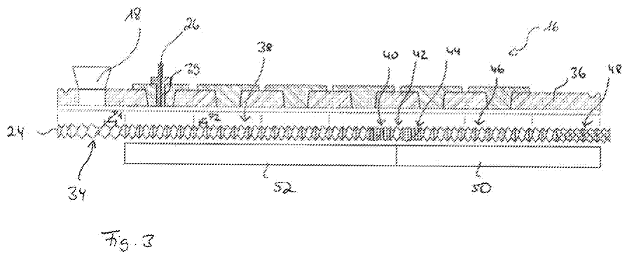

FIG. 3 shows a detailed view of a granulator used in the system for preparing granules in accordance with FIG. 2.

FIG. 1 shows a flow diagram illustrating the main steps of a method for preparing at least partially dried granules. A layout of a system 10 for preparing at least partially dried granules in accordance with the method depicted in FIG. 1 is shown in FIG. 2. The method for preparing at least partially dried granules comprises a step of feeding an API 12, a base powder 14 and a granulation liquid 15 to a granulator 16.

The base powder 14 is provided to the granulator 16 in the form of substantially dry solid powder particles. The base powder 14 contains a binder for powder agglomaration, for example polyvinylpyrrolidone. A selected binder may constitute the only component of the base powder 14. It is, however, also conceivable that the base powder 14 is a binder mixture or a binder containing powder pre-blend, i.e. a binder mixture or a binder containing powder mixture which is prepared in a preceding mixing step. Lactose (spray dried) and/or cellulose, in particular microcrystalline cellulose, may be added so as to serve as tablet binders/diluents. Glyceryl behenate, magnesium stearate, and/or sodium stearyl fumarate may be added as lubricants. Sodium carboxymethyl cellulose may be added as a disintegrant. Further, the dry base powder may contain or consist of solid API powder particles. In particular, the dry base powder may contain powder particles of the same API that is also contained in the slurry fed to the granulator.

As becomes apparent from FIG. 2, the dry base powder 14 is fed to the granulator 16 at a first feeding port 18 of the granulator 16. In particular, the dry base powder 14 is continuously fed to the granulator 16 by means of a powder dosing device 20 which is controlled in dependence on a weight change of a reserve of the dry base powder 14 contained in a reservoir 22 of the powder dosing device 20. Basically, it is conceivable to interrupt the supply of dry base powder 14 for refilling the reservoir 22 of the powder dosing 20 device with the base powder 14. In order to maintain a continuous supply of base powder 14 to the granulator 16, it is, however, preferable to simply interrupt the weight change control and to maintain the feed rate of base powder 14 to the granulator 16 constant, i.e. to operate the powder dosing device 20 “blind” during refilling the reservoir.

In the arrangement depicted in FIG. 2, the granulator 16 is designed in the form of a twin screw extruder, i.e. in the form of a continuous operable extruder device which is equipped with a pair of intermeshing extruder screws 24. The pair of extruder screws 24 is operable to rotate about respective central axes in order to convey an extrudate through the granulator 16 in a conveying direction which in FIG. 2 is indicated by the arrow C.

Further, also the API 12 may be fed to the granulator 16 in the form of substantially dry solid powder particles. In the preferred embodiment of the method for preparing at least partially dried granules as illustrated in the drawings, the API 12, however, is fed to the granulator 16 in the form of a slurry containing solid API powder particles 12 dispersed in a granulation liquid 15.

Basically, the slurry may be prepared by adding a granulation liquid 15 to a previously dried API powder 12. In the preferred embodiment of the method illustrated in the drawings, the granulation liquid 15 contained in the slurry, however, is a washing liquid used for washing solid API powder particles 12 after being synthesized in a liquid phase in a preceding washing step. Hence, the solid API powder particles 12, after being washed, remain dispersed in the washing liquid which then is used as the granulation liquid 15. The granulation liquid 15 contained in the slurry contains water and/or an organic solvent, in particular ethanol. If need be, liquid or solid additives for tailoring slurry properties such as, for example, dispersion of the API particles 12 in the liquid phase 15, wetting of the API particles 12 by the liquid phase 15, viscosity of the slurry, etc. may be added to the slurry. Further, if need be, a granulation liquid in addition to that contained in the slurry may be added to the granulator 16.

The slurry which contains the API 12 and the granulation liquid 15 is fed to the granulator 16 at a second feeding port 26 which, with respect to the conveying direction C, is arranged downstream of the first feeding port 18. In particular, the slurry is fed to the granulator 16 by means of a metered pump 28. In dependence on the type of the API 12 and the properties of the API powder 12 such as, for example, particle size, particle size distribution, surface properties, wetting properties, etc., a concentration of the API 12 within the suspension may be between 10 to 70 weight %. For example, when using ibuprofen as the API, the concentration of the API within the suspension may be between 29 to 53 weight %, when using lumefantrine as the API, the concentration of the API within the suspension may be between 25 to 35 weight %, and when using Opadry yellow as a model API, the concentration of the model API within the suspension may be between 10 to 20 weight %. In any case, in order to ensure a reliable continuous volumetric dosing of the slurry to the granulator 16, the concentration of the API 12 within the suspension, i.e. a liquid/solid ratio within the slurry and a viscosity of the slurry should be appropriately adjusted, if needed by means of the addition of solid or liquid additives to the slurry, in order to allow an unhindered pumping of the slurry into the granulator 16.

A feed rate of the dry powdery base powder 14 to the granulator 16 may vary in dependence on the flow properties of the powdery dry base powder 14, the API load of the slurry, i.e. the liquid/solid ratio within the slurry, the viscosity of the slurry, the liquid/solid ratio within an API/base powder/granulation liquid mixture, the viscosity of the API/base powder/granulation liquid mixture, the wetting behavior of the dry base powder 14 with the granulation liquid 15 contained in the slurry, etc. Typical feed rates of the dry base powder 14 to a small scale granulator 16 may vary between 140 and 500 g/h, whereas typical slurry feed rates to the small scale granulator 16 may vary between 200 and 1000 g/h. Typical feed rates to a medium scale granulator with a 16 mm diameter screw may be up to 10 times higher and typical feed rates to a large scale granulator with a 47 mm diameter screw may be up to 50 kg/h.

Within the granulator 16, the API 12 and the granulation liquid 15 contained in the slurry are mixed with the dry base powder 14. A detailed view of the granulator 16 is depicted in FIG. 3. As becomes apparent from FIG. 3, the extruder screws 24 of the granulator 16 comprise a base powder supply zone 34 for conveying the dry base powder 14 into an extruder housing 36. The base powder supply zone 34 of the extruder screws 24 is substantially aligned with the first feeding port 18 via which the dry base powder 14 is fed to the granulator 16. In the base powder supply zone 34, each of the pair of extruder screws 24 has a first pitch P1 which allows the generation of a suction force by the rotation of the extruder screws 24 so as to convey the dry base powder 14 into the extruder housing 36.

Further, the extruder screws 2 comprise a first compression zone 38 which is adapted to convey and densify the base powder 14 supplied into the extruder housing 36 via the first feeding port 18 before the granulation liquid 15 is added. Hence, in the embodiment of a granulator 16 shown in the drawings, the base powder supply zone 34 and the first compression zone 38 constitute a solid powder particle preprocessing device which is adapted to preprocess the solid powder particles, here the base powder particles 14, within the granulator 16 before the granulation liquid 15 is added.

The first compression zone 38 of the extruder screws 24 is arranged downstream of the base powder supply zone 34 and has a second pitch P2 which is smaller than the first pitch P1. The second feeding port 26 comprises a feeding nozzle 39 which allows the supply of the slurry to the granulator 16 at the desired feed rate. Downstream of the first compression zone 38, the extruder screws 24 comprise a first granulation zone 40 for kneading the API loaded slurry, i.e. the API 12 and the granulation liquid 15, with the dry base powder 14.

In addition, the extruder screws 24 comprise a second compression zone 42 which is arranged downstream of the first granulation zone 40. In the exemplary embodiment of a granulator 16 shown in the drawings, the extruder screws 24, in the second compression zone 42, have a pitch that is equal to the second pitch P2 of the extruder screws 24 in the first compression zone 38 and the design of the extruder screws 24 corresponds to the design of the extruder screws 24 in the first compression zone 38. In the second compression zone 42, the slurry, i.e. the API 12 and the granulation liquid 15 and the base powder 14 are conveyed and further densified. The pitch and the further design of the extruder screws 24 in the second compression zone 42 may, however, be adjusted as needed in dependence on the properties of the API/base powder/granulation liquid mixture, in particular the liquid/solid ratio within the API/base powder/granulation liquid mixture and the viscosity of the API/base powder/granulation liquid mixture.

Further, the extruder screws 24 comprise a second granulation zone 44 for further kneading the slurry, i.e. the API 12 and the granulation liquid 15, with the dry base powder 14. The second granulation zone 44 is arranged downstream of the second compression zone 42.

Moreover, the extruder screws 24 comprise a third compression zone 46 for conveying and further densifying the API/base powder/granulation liquid mixture. The third compression zone 46 is provided on the extruder screws 24 downstream of the second granulation zone 44. In the third compression zone 46, the extruder screws 24 have a pitch that is equal to the second pitch P2 of the extruder screws 24 in the first compression zone 38 and the design of the extruder screws 24 corresponds to the design of the extruder screws 24 in the first compression zone 38 as depicted in FIG. 3b. Like the pitch and the design of the extruder screws 24 in the second compression zone 42, also the pitch and the further design of the extruder screws 24 in the third compression zone 46 may be adjusted as needed in dependence on the properties of the API/base powder/granulation liquid mixture, in particular the liquid/solid ratio within the API/base powder/granulation liquid mixture and the viscosity of the API/base powder/granulation liquid mixture.

Finally, although the slurry/base powder mixture should already be homogenized upon exiting the second granulation zone 44, the extruder screws 24 comprise a homogenization zone 48 for further homogenizing the API/base powder/granulation liquid mixture. The homogenizing zone 48 is arranged downstream of the third compression zone 46 and the extruder screws 24, in the homogenizing zone 48, are designed in the form of a distributive feed screw which provides for a final homogenization of the API/base powder/granulation liquid mixture prior to exiting the granulator 16.

It should be acknowledged, that the dimensions, i.e. the lengths of the different zones 34, 38, 40, 42, 44, 46, 48 of the extruder screws 24 as well as the design of the different zones 34, 38, 40, 42, 44, 46, 48 of the extruder screws 24 may be varied as needed in dependence on the properties of the API 12, the dry base powder 14, the granulation liquid 15 and the API/base powder/granulation liquid mixture. The zones 40, 42, 44, 46, 48 of the extruder screws 24 which are arranged downstream of the second feeding port 26 in combination form a mixing device for mixing the API 12, the base powder 14 and the granulation liquid 15 within the granulator 16 in order to produce an API/base powder/granulation liquid mixture. Further, the extruder screws 24 may be cooled, at least in the region of the first and the second granulation zone 40, 44, e.g. by means of cooling channels provided in a housing of the granulator 16.

The granulator 16 further comprises a heating device 50 for heating the API/base powder/granulation liquid mixture within the granulator 16 to a heating temperature T_{H} that exceeds an evaporation temperature T_{E} of the granulation liquid 15 contained in the API/base powder/granulation liquid mixture. In particular, the heating device 50 heats the API/base powder/granulation liquid mixture to a heating temperature T_{H} that is between 1.5 to 2.5 times the evaporation temperature T_{E} of the granulation liquid 15 for a heating time of <15 seconds, preferably <10 seconds, in particular <6 seconds. As a result of the operation of the heating device 50, at least a part of the granulation liquid 15 contained in the API/base powder/granulation liquid mixture evaporates, wherein the heating time should be kept as short as possible in order to avoid a deterioration of the API 12.

Further, the granulator 16 comprises a preheating device 52 which is adapted to preheat the API/base powder/granulation liquid mixture, prior to being heated to the heating temperature T_{H}, to a preheating temperature Tₚ that is lower than the heating temperature T_{H}. In particular, the preheating device 52 preheats the API/base powder/granulation liquid mixture to a preheating temperature Tₚ that is between 0.2 to 1.0 times the evaporation temperature T_{E} of the granulation liquid 15 contained in the API/base powder/granulation liquid mixture for a preheating time of <15 seconds, preferably <10 seconds, in particular <6 seconds. Preferably, the preheating time is longer than the heating time in order to allow a smooth heating and hence evaporation process to take place.

The heating device 50 and/or the preheating device 52 may be arranged outside of the extruder housing 36 and hence heat the API/base powder/granulation liquid mixture from outside. It is, however, also conceivable to integrate the heating device 50 and/or the preheating device 52 into the extruder housing 36.

As becomes apparent from FIG. 3, the heating device 50 is extends in the region of a part of second compression zone 42, the second granulation zone 44, the third compression zone 46 and in the region of the homogenization zone 48 of the extruder screws 24, whereas the preheating device 52 extends in the region of the first compression zone 38, in the region of the first granulation zone 40 and in the region of a part of the second compression zone 42 of the extruder screws 24. Hence, the base powder supply zone 34 constitutes a processing device which is adapted to process, i.e. mix, the API/base powder/granulation liquid mixture prior to being heated, in particular for a processing time of 0 to 10 seconds. The processing time may be determined and adjusted in dependence on the amount of solid particles contained in the API/base powder/granulation liquid mixture. For adjusting the processing time, the position and the lengths of the heating device 50 and the preheating device 52 relative to the extruder screws 24 and/or the conveying speed of the API/base powder/granulation liquid mixture through the granulator 16 may be varied.

The operation of the heating device 50 and the operation of the preheating device 52, like the operation of the extruder screws 24, the operation of the pump 28 and the operation of the powder dosing device 20, are controlled by means of a central control unit 60 of the granulator 16. By means of the control unit 60, the heating temperature T_{H} and preheating temperature Tₚ can be controlled as desired. Further, the control of the supply of API loaded slurry and dry base powder 14 is made possible. Finally, by controlling an operating speed of the extruder screws 24, a conveying speed of the dry base powder 14 and the API/base powder/granulation liquid mixture through the granulator 16 and hence a residence time of the dry base powder 14 and the API/base powder/granulation liquid mixture within the individual sections of the granulator 16 as well as the heating time and the preheating time of the API/base powder/granulation liquid mixture can be controlled.

Preheating and heating of the API/base powder/granulation liquid mixture leads to a drying of the API/base powder/granulation liquid mixture already within the granulator 16. Consequently, at least partially dried granules 56 are discharged from the granulator 16 via a discharge opening 58 of the granulator 16, wherein in the embodiment of a granulator 16 depicted in the drawings, the extruder screws 24 serve as a conveying device for discharging the at least partially dried granules 56 from the granulator 16. Typical dried granule sizes vary from 200 to 1000 μm.

The granulation liquid 15 evaporating during heating of the API/base powder/granulation liquid mixture is removed from the granulator 16 via an evaporation opening 54 which is formed separate from the discharge opening 58 for discharging the at least partially dried granules 56 from the granulator 16 and separate from the feeding ports 18, 26 for feeding the base powder 14 and the API 12 together with the granulation liquid 15 to the granulator 16. As a result, unhindered removal of evaporated granulation liquid 15 from the granulator 16 is ensured even during continuous operation of the granulator 16, i.e. even during continuous supply of slurry and dry base powder 14 to the granulator 16. The granules 56 exiting the discharge opening 58 of the granulator 16 thus can further be processed without time-consuming further drying in a separate drying equipment.

The at least partially dried granules 56 obtained via the above-described method may be suitable for direct oral dosage or may be intended to be filled into capsules. Preferably, however, the granules 56 are compacted so as to form tablets.

### EXAMPLE 1

A granulation process with microcrystalline cellulose as dry solid powder particles and water as granulation liquid was performed using the process parameters summarized in table 1 .

TABLE 1
Example 1 Microcrystalline cellulose + water

| Factor | Range | |
|---|---|---|
| Total material mass flow | 300-730 | g/h |
| rate (solid flow + liquid | | |
| flow) | | |
| L/S ratio | 0.33-1.00 | |
| (liquid/solid ratio) | | |
| Screw speed | 200-800 | rpm |
| Preheating temperature | 20-100° | C. |
| Heating temperature | 160-200° | C. |

Equipment setup

| | |
|---|---|
| Twin screw granulator | Thermo Fisher 11 MM Pharma |
| type: | |
| Solid feed position: | base powder supply zone |
| Granulation liquid feed | first compression zone |
| position: | |
| Preheating zones: | first compression zone, |
| | first granulation zone, |
| | second compression zone, |
| Heating and evaporation | second compression zone, |
| | second granulation zone, |
| | third compression |
| zones: | zone, homogenization zone |

Twin-screw wet granulation was performed on a co-rotating Pharma 11 mm twin-screw granulator (Thermo Scientific Pharma 11, Thermo Fisher Scientific, Karlsruhe, Germany). The twin-screw granulator was operated at varying screw speeds of 200-800 rpm, varying total material mass flow rates of 300-730 g/h and varying L/S ratios of 0.33-1.00. The powder component cellulose was fed into the twin-screw granulator using a Brabender gravimetric feeder (DDW-MDO-MT-1, Brabender, Duisburg, Germany) in the region of an base powder supply zone of the granulator screws. The granulation liquid was dosed to the twin-screw granulator using a progressing cavity pump (NM003BY11S12B, Netzsch Group, Selb, Germany) in the region of a first compression zone of the granulator screws. Preheating was performed in a first compression zone, a first granulation zone and a second compression zone of the granulator screws, whereas heating was performed in the second compression zone, a second granulation zone, a third compression zone and a homogenization zone of the granulator screws. The preheating temperature was varied between 20-100° C. and the heating temperature was varied between 160-200° C.

It was shown that dried or partially dried material was obtained after the granulation process with an integrated in situ drying step. Furthermore, it has been shown that the varied process parameters, i.e. the total mass material flow rate, the L/S ratio, the screw speed, the preheating temperature, and the heating temperature influence the fraction of granulation liquid which is evaporated from the granulator and hence removed from the liquid/solid mixture within the granulator.

### EXAMPLE 2

A granulation process was performed using Lumefantrine as a model drug (API) substance, while other commonly used base powders for pharmaceutical wet granulation were added to the formulation. Water was used as the granulation liquid. The process parameters are summarized in table 2.

TABLE 2
Example 2: Lumefantrine as model drug substance

| | | Concentration |
|---|---|---|
| Material | Role | (% w/w) |
| Lumefantrine | drug substance | 20.0 |
| Cellulose, microcrystalline | filler | 33.5 |
| Lactose | filler | 35.0 |
| Polyvinylpyrrolidone | binder | 4.0 |
| Sodium Carboxy Methyl | disintegrant | 6.0 |
| Cellulose | | |
| Magnesium stearate | lubricant | 1.5 |

Equipment setup

| | |
|---|---|
| Twin screw granulator | Thermo Fisher 11 MM Pharma |
| type: | |
| Solid feed position: | base powder supply zone |
| Granulation liquid feed | first compression |
| position: | zone |
| Preheating | first compression zone, |
| zones: | first granulation zone, |
| | second compression zone |
| Heating and | second compression zone, |
| evaporation | second granulation zone, |
| | third compression |
| zones: | zone, homogenization zone |

| Factor | Range | |
|---|---|---|
| Total material mass flow | 240-630 | g/h |
| rate (solid flow + liquid | | |
| flow) | | |
| L/S ratio | 0.5-1.00 | |
| (liquid/solid ratio) | | |
| Screw speed | 40-500 | rpm |
| Preheating temperature | 20-110° | C. |
| Heating temperature | 160-220° | C. |

Twin-screw wet granulation was performed on a co-rotating Pharma 11 mm twin-screw granulator (Thermo Scientific Pharma 11, Thermo Fisher Scientific, Karlsruhe, Germany). The twin-screw granulator was operated at varying screw speeds of 50-500 rpm, varying total material mass flow rates of 240-500 g/h and varying L/S ratios of 0.63-1.00. The model drug substance and the base powders were fed into the twin-screw granulator using a Brabender gravimetric feeder (DDW-MDO-MT-1, Brabender, Duisburg, Germany) in the region of an base powder supply zone of the granulator screws. The granulation liquid was dosed to the twin-screw granulator using a progressing cavity pump (NM003BY11S12B, Netzsch Group, Selb, Germany) in the region of a first compression zone of the granulator screws. Preheating was performed in a first compression zone, a first granulation zone and a second compression zone of the granulator screws, whereas heating was performed in the second compression zone, a second granulation zone, a third compression zone and a homogenization zone of the granulator screws. The preheating temperature was varied between 20-110° C. and the heating temperature was varied between 160-220° C.

It was shown that completely dried granules or partially dried granules can be obtained for a pharmaceutically relevant formulation via the granulation process with an integrated in situ drying step.

1. A method for preparing at least partially dried granules (56), the method comprising:
feeding an API (12), a base powder (14) and a granulation liquid (15) to a granulator (16),
mixing the API (12), the base powder (14) and the granulation liquid (15) within the granulator (16) in order to produce an API/base powder/granulation liquid mixture, and
heating the API/base powder/granulation liquid mixture within the granulator (16) to a heating temperature (T_{H}) that exceeds an evaporation temperature (T_{E}) of the granulation liquid (15) contained in the API/base powder/granulation liquid mixture so as to allow at least a part of the granulation liquid (15) contained in the API/base powder/granulation liquid mixture to evaporate in order to obtain at least partially dried granules (56) containing at least the API (12) and the base powder (14), and
discharging the at least partially dried granules (56) from the granulator (16).

2. The method according to claim 1, wherein the API/base powder/granulation liquid mixture is heated to a heating temperature (T_{H}) that is 1.5 to 2.5 times the evaporation temperature (T_{E}) of the granulation liquid, and/or wherein the API/base powder/granulation liquid mixture is heated to the heating temperature (T_{H}) for a heating time of <15 seconds, preferably <10 seconds, and in particular <6 seconds.

3. The method according to claim 1, wherein the API/base powder/granulation liquid mixture, prior to being heated to the heating temperature (T_{H}), is preheated to a preheating temperature (Tₚ) that in particular is between 0.2 and 1.0 times the evaporation temperature (T_{E}) of the granulation liquid contained in the API/base powder/granulation liquid mixture.

4. The method according to claim 3, wherein the API/base powder/granulation liquid mixture is preheated to the preheating temperature (Tₚ) for a preheating time of <15 seconds, preferably <10 seconds, and in particular <6 seconds, the preheating time in particular being longer than the heating time.

5. The method according to claim 1, wherein the granulation liquid (15) that evaporates from the API/base powder/granulation liquid mixture is discharged from the granulator (16) via an evaporation opening (54) that is formed separate from a discharge opening (58) for discharging the at least partially dried granules (56) from the granulator (16) and separate from (a) feeding port(s) (18, 26) for feeding the API (12), the base powder (14) and/or the granulation liquid (15) to the granulator (16).

6. The method according to claim 1, wherein the API/base powder/granulation liquid mixture, prior to being heated, is processed for a processing time of 0 to 10 seconds, the processing time in particular being determined in dependence on the amount of solid particles contained in the API/base powder/granulation liquid mixture.

7. The method according to claim 1, wherein at least one of the API (12) and the base powder (14) is fed to the granulator (16) in the form of substantially dry solid powder particles.

8. The method according to claim 1, wherein the API (12) is fed to the granulator (16) in the form of a slurry containing solid API powder particles dispersed in a liquid, the liquid contained in the slurry in particular containing a synthesizing liquid used for synthesizing the solid API powder particles in a preceding API synthesizing step or a washing liquid used for washing the solid API powder particles in a preceding washing step.

9. The method according to claim 8, wherein at least a part of the granulation liquid (15) fed to the granulator (16) consists of the liquid used for preparing the slurry containing solid API powder particles (12).

10. The method according to claim 1, wherein at least one of the API (12) and the base powder (14) is fed to the granulator (16) in the form of substantially dry solid powder particles upstream of the granulation liquid (15), the dry solid powder particles in particular being preprocessed within the granulator (16) before the granulation liquid (15) is added.

11. At least partially dried granules (56) prepared by a method according to claim 1.

12. A method for manufacturing solid oral dosage forms, comprising:
preparing at least partially dried granules (56) in accordance with the method according to claim 1, and
compacting the granules (56) so as to form tablets or filling the granules (56) into capsules.
